(19) United States
(12) Patent Application Publication
(10) Pub. No.: US 2023/0251186 A1
(43) Pub. Date: Aug. 10, 2023

(54) METHOD AND APPARATUS FOR LONG TERM ACCURATE MEASUREMENT OF AMMONIA GAS CONCENTRATION IN A PERMANENT AMMONIA GAS ENVIRONMENT

(71) Applicant: TECHNOLOGIES INTELIA INC., Montreal (CA)

(72) Inventors: Jacques Godin, Quebec, QC (CA); Claude Bouchard, Lanoraie, QC (CA); Josee Samson, Lanoraie, QC (CA)

(21) Appl. No.: 17/906,680

(22) PCT Filed: Mar. 19, 2021
(86) PCT No.: PCT/CA2021/050371

§ 371 Date: Sep. 19, 2022

Related U.S. Application Data
(60) Provisional application No. 62/991,885, filed on Mar. 19, 2020.

(51) Int. Cl. G01N 21/33; G01N 21/61

(52) U.S. Cl. CPC G01N 21/33; G01N 21/61

(57) ABSTRACT

A method and apparatus are described for measuring the concentration of a gas with an absorption band in the ultraviolet range. The device includes an absorption chamber containing a gas, a light source, a selected optical bandpass filter, and ultraviolet photodetectors. The gas concentration is measured by the ratio of a transmitted intensity to an incident intensity with the Beer-Lambert Law relation. A second light source may be used for a compensation signal. A second method periodically changes the absorption coefficient by inserting a transparent material in the absorption path to measure the optical compensation signal. A third method periodically shortens the optical absorption path by moving the active detector closer to the light source to measure the optical compensation signal. The fourth method uses an optical element to deflect the optical beam to create a shorter absorption path as a reference for the incident signal using one detector.

### CROSS-REFERENCE TO RELATED APPLICATIONS

The present patent application claims the benefits of priority of American Pat. Application No. US 62,991,885, entitled “METHOD AND APPARATUS FOR LONG TERM ACCURATE MEASUREMENT OF AMMONIA GAS CONCENTRATION IN A PERMANENT AMMONIA GAS ENVIRONMENT” and filed at the United States Patent and Trademark Office on Mar. 19, 2020, the content of which is incorporated herein by reference.

### FIELD OF THE INVENTION

The present invention generally relates to method and systems for measuring gas, more specifically to methods and systems for measuring ammonia gas concentration using ultraviolet gas analysers.

### BACKGROUND OF THE INVENTION

Manure in livestock buildings generates ammonia and other toxic gases that affect the health of animals and production staff when the gas concentration reaches and uncontrolled level. Typical use of an ammonia sensor in the barns aims to regulate building ventilation to maintain toxic gas concentration below the maximum gas toxicity exposure limit.

There are many technologies available to measure the ammonia concentration, but no affordable solution exists with a reasonable lifetime and capable of sustain in a constant ammonia environment without loss of accuracy requiring frequent replacement of the measuring element.

Excess levels of ammonia can be dangerous to the health of animal and can widely affect the growing conditions. To warn the farmer of a high concentration of ammonia in the barn’s atmosphere, ammonia level is monitored using an ammonia gas sensor. Environmental conditions in the barns are special by the fact that the concentration of ammonia is almost always between 10 to 40 ppm. Conventional electrochemical type gas sensors are not suitable for those environments by the fact that the chemical-cell cannot be constantly immersed in an environment which is high in ammonia concentration; the lifetime of the chemical cell is greatly reduced so an electrochemical-type gas sensor does not offer and effective solution for this kind of application.

In recent years, optical based gas analysers have been introduced in the marketplace with the bulk of product utilizing infrared wavelengths to measure gas concentration. IR analysers typically have a high measuring accuracy and sensitivity and a high selectivity like photoacoustic based sensor or TDLA spectroscopy, but they are very expensive and not suitable for farming industry. Other NDIR low-cost sensors have been developed for the mass market like CO₂, CO and SO₂ sensor, but they suffer from poor accuracy with a range of ± 30 ppm that is far away from what the farming industry need.

Many toxic gases absorb ultraviolet radiation in a 190-330 nm wavelength region. The absorption cross section in the ultraviolet band if mostly two orders of magnitude higher than the one in the NIR band. Based on these assumptions, we present an invention to provide an ultraviolet gas analyser capable of measuring the concentration of toxic gas in a harsh atmospheric environment constantly filled with high concentration of ammonia and others toxic gas.

### SUMMARY OF THE INVENTION

The aforesaid and other objectives of the present invention are realized by generally providing a method and an apparatus for measuring the concentration of gas interacting with ultraviolet light

In an aspect of the present invention, a system for measuring concentration of a gas is provided. The system comprises an absorption chamber comprising an air inlet and an air outlet, an active photodetector within the absorption chamber to measure a light radiation, a light source emitting an ultraviolet radiation within an absorption spectrum of the gas along an absorption path toward the active photodetector, an optical bandpass filter between the light source and the active detector and a reference photodetector positioned to measure the light radiation of the light source entering in the absorption chamber.

The light source may be a controllable ultraviolet “arc type”.

The system may further comprise a first interface for sampling a signal outputted by the active photodetector, a second interface for sampling the signal outputted by the reference photodetector and a computerized device connected to the first and second sampling interfaces, the computerized device being configured to calculate the concentration of the gas as a function of light absorption based on a ratio of the output signals of the reference photodetector and of the active photodetector.

The system may further comprise an optical element between the light source and the absorption chamber used to generate a collimated light beam within the absorption chamber. The system may further comprise a lens adjacent to the active detector for collimating the light emitted in the absorption chamber to the active photodetector. The system may further comprise a heating element adapted to heat the optical bandpass filter at a temperature higher than the dew point temperature of the gas.

The system may further comprise a beam splitter to direct a portion of the light radiation emitted in the absorption chamber towards the reference detector. The system may further comprise a controllable reference light source emitting a light radiation outside of the absorption spectrum of the gas, the light radiation being measurable by the active and the reference photodetector. The system may further comprise a drift compensation mechanism. The drift compensation mechanism may be configured to adjust the measurement of the gas concentration using a proportional ratio of drift values measured by the active photodetector and the reference photodetector since the last calibration.

In another aspect of the invention, a system for measuring concentration of a gas is provided. The system comprises an absorption chamber comprising an air inlet and an air outlet, an active photodetector within the absorption chamber to measure light radiation, a controllable light source emitting an ultraviolet light beam within an absorption spectrum of the gas along an absorption path toward the active photodetector, an optical bandpass filter between the light source and the active detector; and a device to change the length of the light path in the absorption chamber between the light source to the active photodetector.

The system may further comprise a first interface for sampling the ultraviolet light beam detected by the active photodetector, a second interface for controlling position of the device to change the length of the light path within the absorption chamber and a computerized device connected to the first and the second interfaces, the computerized device being configured to calculate the concentration of the gas as a function of light absorption measured by the signal ratio before and after changing the length of the light path through the absorption chamber.

The device to change the length of the light path may be a light pipe being insertable in the light path yet removable from the light path between the light source and the active photodetector.

The device to change the length of the light path between the light source and the active photodetector may be a support movable toward and away from the light source, the active photodetector being mounted to the movable support. The support may be moved using an electromotive force. The support may be a carriage. The support may comprise two mating portions, the first portion slidingly moving within the second portion to change the length of the light path.

The light source and the active photodetector may be oriented in the same direction toward the absorption chamber. The device to change the length of the light path may comprise a first reflecting member in the absorption chamber returning the ultraviolet light beam to the active photodetector setting a long light path and a second reflecting member insertable between the first reflecting member and the light source to set a short light path.

The system may further comprise an optical element between the light source and the absorption chamber used to generate a collimated light beam within the absorption chamber. The system may further comprise a lens adjacent to the active detector for collimating the light emitted in the absorption chamber to the active photodetector. The system may further comprise a heating element adapted to heat the optical bandpass filter at a temperature higher than the dew point temperature of the gas.

In yet another aspect of the invention, a method for measuring a concentration of a gas present in an absorption chamber is provided. The method comprises emitting a light beam through the absorption chamber at a wavelength absorbed by the gas, measuring a reference intensity of the emitted light entering in the absorption chamber, measuring an active intensity of the emitted light after passing through the gas in the absorption chamber at a predetermined distance of the emission of the light and calculating the gas concentration based on the ratio of the of measured active intensity and of the measured reference intensity.

The method further may further comprise filtering the emitted light entering the absorption chamber at a wavelength absorbed by the gas. The measuring of the reference intensity may be performed by a first photodetector and the measuring of the active intensity being performed by a second photodetector.

The method may further comprise deflecting a portion of the emitted light to measure the reference intensity.

In another aspect of the invention, a method for measuring a concentration of a gas present in an absorption chamber comprising a light path having a variable length between a light source and a photodetector is provided. The method comprises reducing the length of the light path in the absorption chamber, emitting a light beam through the absorption chamber at a wavelength absorbed by the gas in the reduced light path, measuring a reference intensity of the emitted light in the reduced light path, increasing the length of the light path in the absorption chamber, emitting the light beam through the absorption chamber at a wavelength absorbed by the gas in the increased light path, measuring an active intensity of the emitted active light beam in the increased light path and calculating the gas concentration based on the ratio of the measured intensities from the reduced light path and the increased light path.

The reducing of the length of the light path in the absorption chamber may further comprise inserting into the emitted light beam a light pipe inert to the gas. The reducing of the length of the light path in the absorption chamber may further comprise moving the light source and the photodetector toward one another.

The photodetector and the light source may be oriented in the same direction, the photodetector receiving the light beam through a first reflecting member, the reducing of the length of the light path in the absorption chamber further comprising placing a second reflecting member between the light source and the first reflecting member.

In a further aspect of the present invention, a method to correct for short and long terms drifts of the system is provided. The method further comprises turning off all of the active light source and the reference light source, measuring a reference intensity when the active light source and the reference light source are turned off, measuring an active intensity when the active light source and the reference light source are turned off, turning on the reference light source through the absorption chamber at a wavelength outside of the absorption spectrum of the gas, measuring a reference intensity when the reference light source is turned on, measuring an active intensity when the active light source is turned on, calculating a reference signal drift based on the difference between the measured reference intensities, calculating an active signal drift based on the difference between the measured active intensities, calculating a drift ratio of the reference signal drift and the active signal drift and correcting calculation of the gas concentration using the calculated drift ratio.

The features of the present invention which are believed to be novel are set forth with particularity in the appended claims.

### BRIEF DESCRIPTION OF THE DRAWINGS

The above and other objects, features and advantages of the invention will become more readily apparent from the following description, reference being made to the accompanying drawings in which:

FIG. 1 is an optical diagram of a first embodiment of an ultraviolet toxic gas analyser in accordance with the principles of the present invention.

FIG. 2A is an optical diagram of a second embodiment of an ultraviolet toxic gas analyser in accordance with the principles of the present invention.

FIG. 2B is another optical diagram of the ultraviolet toxic gas analyser of FIG. 2A.

FIG. 3A is showing the details of a first side of an actuator for an UV-light pipe in accordance with the principles of the present invention

FIG. 3B is showing the details of a second side of an actuator for an UV-light pipe in accordance with the principles of the present invention.

FIG. 4A is an optical diagram of a third embodiment of an ultraviolet toxic gas analyser in accordance with the principles of the present invention.

FIG. 4B is another optical diagram of the ultraviolet toxic gas analyser of FIG. 4A.

FIG. 4C is an optical diagram of an embodiment of a device to change the length of the light path in a gas analyser in accordance with the principles of the present invention.

FIG. 5A is an optical diagram of a fourth embodiment of an ultraviolet toxic gas analyser in accordance with the principles of the present invention.

FIG. 5B is another optical diagram of the ultraviolet toxic gas analyser of FIG. 5A.

FIG. 5C is yet another optical diagram of the ultraviolet toxic gas analyser of FIG. 5A.

FIG. 6A is showing a rotating blade when measuring the incident signal in accordance with the principles of the present invention.

FIG. 6B is showing a rotating blade out of the path of an optical beam in accordance with the principles of the present invention.

FIG. 6C is showing a rotating blade when an attenuation element is front of an optical beam in accordance with the principles of the present invention.

FIG. 7 is a graphic illustrating the absorption cross section of ammonia in an ultraviolet band in accordance with the principles of the present invention.

FIG. 8 is showing a high-level block diagram of each subpart of a gas analyser attached to a microcontroller in accordance with the principles of the present invention.

FIG. 9 is showing a high-level block diagram of each subpart of a gas analyser attached to a microcontroller in accordance with the principles of the present invention.

FIG. 10 is showing the detailed aspect of an ultraviolet light source in accordance with the principles of the present invention.

FIG. 11 is showing the detailed aspect of a reference and active detector in accordance with the principles of the present invention.

FIG. 12 is illustrating a timing diagram of sampling and acquisition sequences when using a 380 nm LED as a compensation signal in accordance with the principles of the present invention.

FIG. 13 is illustrating a timing diagram of sampling and acquisition sequences when using a UV-light pipe to generate a compensation signal in accordance with the principles of the present invention.

FIG. 14 is illustrating a timing diagram of sampling and acquisition sequences when moving an active detector to generate a compensation signal in accordance with the principles of the present invention.

FIG. 15 is illustrating a timing diagram of sampling and acquisition sequences when an optical element is in an optical path to create a short absorption path in accordance with the principles of the present invention.

FIG. 16 is an illustration of different flow diagrams of the calibration process depending on selected embodiments in accordance with the principles of the invention.

### DETAILED DESCRIPTION OF THE PREFERRED EMBODIMENT

A novel method and apparatus for measuring the concentration of gas interacting with ultraviolet light will be described hereinafter. Although the invention is described in terms of specific illustrative embodiment(s), it is to be understood that the embodiment(s) described herein are by way of example only and that the scope of the invention is not intended to be limited thereby.

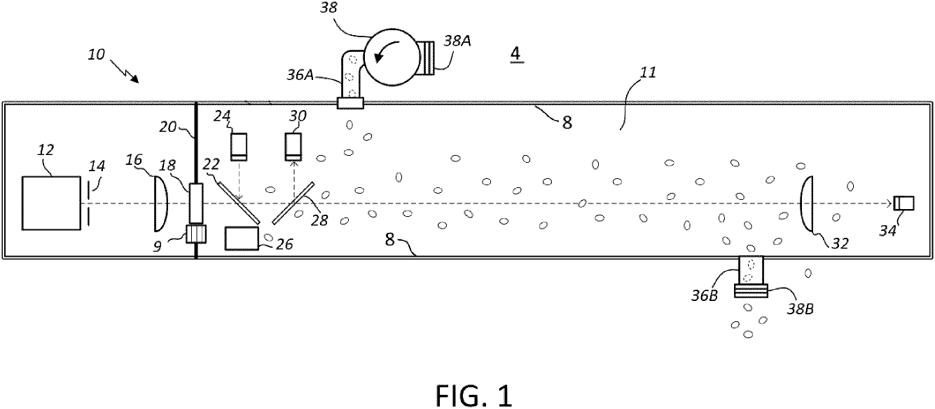

As indicated above, a method and an apparatus for measuring the concentration of gas interacting with ultraviolet light is disclosed. In a preferred embodiment, the measured gas is a toxic gas such as ammonia. Understandably, other toxic gases may also be considered. As shown in FIG. 1, the gas analyser 10 includes an absorption chamber 11. The absorption chamber 11 comprises an inlet 36A connected to receive air, ammonia, or other gas, from a monitored area 4, generally external to the chamber 11, by means of a pump 38 and/or a semipermeable membrane 38A. The absorption chamber 11 is typically part of the structural body of the instrument. In some embodiments, the inside walls 8 of the absorption chamber 11 are made with non-reflective material, such as but not limited to ABS or anodized aluminum. After passing inside the absorption chamber 11, the gas is expulsed towards an outlet 36B which may further comprise a particle filter 38B at the end. The semipermeable membrane 38A and the filter 38B generally aim at allowing ambient gases to enter and leave the sample gas absorption chamber 11 freely, and to avoid particles of dust, smoke, and any other unwanted particles from entering the absorption chamber 11.

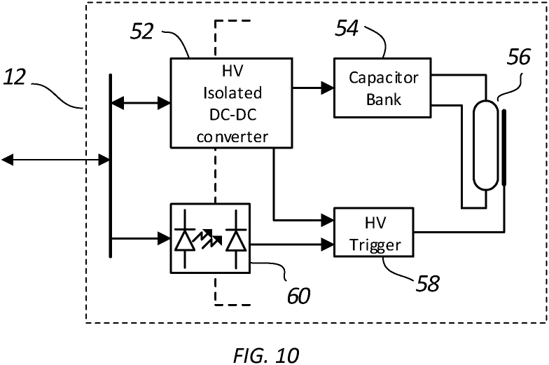

Referring to FIG. 10, an ultraviolet light source’s electrical diagram 12 is illustrated. The ultraviolet light source 12 provides microsecond pulse duration, high peak power of light from ultraviolet to near infrared. The ultraviolet radiation is provided by a short arc length xenon flash lamp 56 generally made with fused silica or quartz glass transparent to wavelength radiations down to 190 nm. A high voltage capacitor bank 54 is charged above the voltage level required to provide a current density in the xenon flash lamp 56 high enough to emit ultraviolet radiation down to 190 nm. The capacitor bank 54 is typically made with high dielectric constant material such as but not limited to polyester film or ceramic, for this exemplary embodiment. The flashlamp 56 is discharged at a rate determined by the control algorithm using the trigger 58 via the galvanically isolated circuit 60. In such embodiment, both the capacitor bank 54 and the trigger 58 are supplied with a high voltage isolated DC-DC converter 52. The galvanic isolation circuit 60 may be a magnetic transformer, a capacitive circuit, a hall-effect circuit, or any other type of isolated circuit that can transmit low skew logical signal. For this exemplary embodiment, the galvanic isolated circuit 60 is made with an optocoupler. Galvanic isolation circuit and isolated DC-DC converter generally help to prevent coupling of common-mode noise into the highly sensitive analog circuits like the reference 30 and active detector 34.

Referring back to FIG. 1, the broadband light emitted from the UV light source 12 is then collimated by a lens 16 made of an ultraviolet-transparent material, such as but not limited to UV fused silica-based glass and may be a plano-convex F4 lens having a 20 mm focal length, for this exemplary embodiment. A small pinhole 14 of a fraction of millimeters in diameter in front of the xenon lamp source 56 generally aims at reducing the divergence of the collimated beam.

The broadband collimated optical beam passes through a narrow bandpass filter 18 so that the UV emitted radiation is in accordance with a wavelength that is strongly absorbed by the gas whose concentration is to be determined. In some embodiments, the system 10 comprises an optical shield 20 made with a non-transparent material. The optical shield 20 is typically used to encloses the bandpass filter 18 to avoid broadband emission in the absorption chamber 11. For this exemplary embodiment, the bandpass filter 18 may have a centered wavelength of 200 nm ± 3 nm and a bandwidth of 10 nm ± 2 nm FWHM chosen to interact with ammonia absorption lines. Other filters center wavelength and bandwidth in the ultraviolet may be used depending on the gas and the concentration, within the scope of this invention.

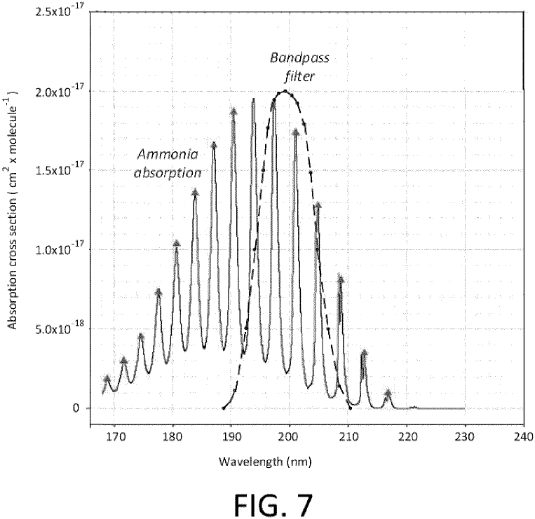

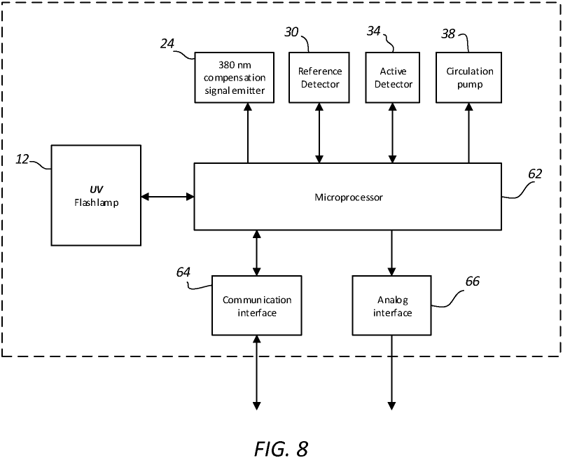

FIG. 7 shows an example of the absorption cross section of ammonia in the VUV-UV band. In this example, the ammonia absorption lines overlap the selected bandpass filter described above. It is obvious to see that absorption propriety of ammonia in the ultraviolet band is much greater (two order of magnitude) than the one in the infrared band (IR). Furthermore, no cross sensitivity with these common gases (CO2, O2, CO, CH4 and water vapor) have been observed in the (180 - 230 nm) band. These properties give a significant advantage to this invention over classic NDIR or TLDAS technologies in the IR band.

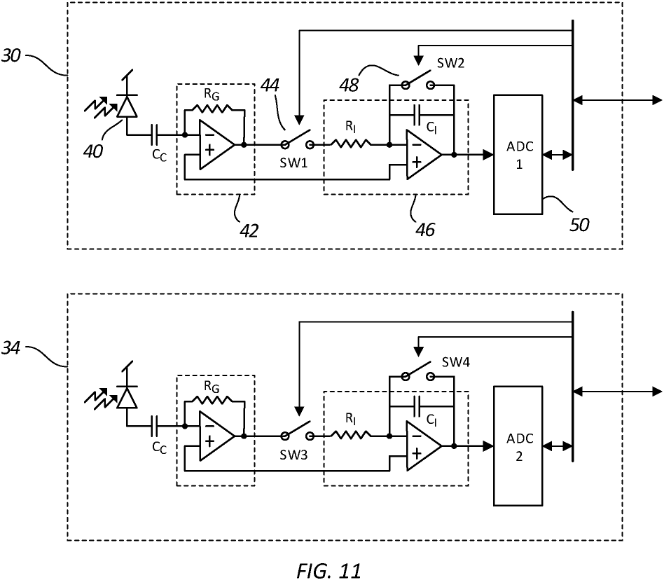

Referring back to FIG. 1, the highly collimated narrowband UV radiation, also referred as the incident light, enters the gas absorption-chamber 11 which is filled with the gas to be analysed. In such embodiment, the incident light is sampled using a beam splitter 28 and a photodetector 30, also referred as a reference detector, illustrated in FIG. 11. The photodetector 30 may be any suitable light sensitive device but, in the illustrated embodiment, is an ultraviolet enhanced silicon photodiode having a response of 0.05 A/W in the band of interest. The intensity measured by the reference detector 30 is substantially proportional to the incident light entering the absorption chamber 11. The beam splitter 28 is made of ultraviolet transmitting material, with or without coated surfaces, such as UV fused silica. It may have the form of a right-angle prism, a pentaprism or other geometrical forms that split the incident light in two beams. For example, and as a preferred embodiment, the beam splitter 28 is made with a 1.5 mm thickness uncoated UV fused silica window having 4% of reflectivity on each surface.

The light travels along the absorption path and reach the collimation lens 32 located at the end of the absorption chamber that collects and focused the light to a second photodetector 34, also referred to as the active detector. The active detector 34 is almost identical to the reference detector 30 describe above, both are illustrated in FIG. 11. The lens 32 is generally made of an ultraviolet-transparent material, such as but not limited to UV fused silica-based glass and may be a plano-convex F1.4 lens with 35 mm focal length, for this exemplary embodiment. The intensity measured by the active detector 34 is substantially proportional to the transmitted light across the absorption chamber and now refers to the transmitted intensity.

The transmitted intensity I_{T} is related to incident light said the incident intensity I_{R}, by the Beer-Lambert law:

$$\text{IT} = {\text{ρ}}_{0} {\text{I}}_{\text{R}} \text{e-} \text{σ} \text{n L}$$

where σ is the absorption cross-section of the gas at a particular wavelength in cm²/molecule, n is the volume number density of the gas in molecule/cm³ and L the length of the optical absorption path in cm.

Since the flashlamp spectrum is not flat in the UV band, the narrow band filter 18 may have a non-rectangular shape, the absorption cross-section σ is a function of the wavelength and the optical absorption path length may vary, we define the coefficient K = σL and replace n in unit of number density by C the concentration in unit of ppm. The coefficient K is obtained at the time of calibration with a gas of known concentration C. The Beer-Lambert law applied to calculate the gas concentration based on the ratio of the transmitted intensity I_{T} to the incident intensity I_{R} can be rewritten as follow.

$$\text{IT} = {\text{ρ}}_{0} {\text{I}}_{\text{R}} \text{e-KC}$$

The correction factor ρ0 is calculated under 0 ppm of gas concentration C and it is the ratio of the UV transmitted intensity at 0 ppm, I_{T0}, measured by the active detector 34 to the UV incident intensity at 0 ppm, I_{R0}, measured by the reference detector 30.

$${\text{ρ}}_{0} = {\text{I}}_{\text{T0}} / {\text{I}}_{\text{R0}}$$

The correction coefficient ρ₀ generally compensates for the inherent differential optical intensity of UV light falling on the reference 30 and active 34 detectors at 0 ppm gas concentration and compensates for the analog gain difference of the reference 30 and active 34 detector. The absorption path length between the beam splitter 28 and the said active detector can be extended in an advantageous manner to increase the absorption and the sensitivity of the gas analyser apparatus.

In another aspect, this invention includes a method to compensate for short term drifts due to temperature change and long-term drifts namely due to components aging. The method uses a gas analyser which does not require further calibration and where the measurement of the gas concentration is very stable over different temperature levels. The reference 30 and active 34 detector circuits comprise one or more photodiode. The photodiodes are sensitive to temperature variations, similarly to other semiconductor devices. For example, the temperature coefficient of a typical UV enhanced photodiode is 0.1%/°C. By comparison, the ultraviolet light attenuation in the presence of ammonia in the absorption chamber is 0.075%/ppm for a 10 cm absorption path length. A small difference of the temperature-coefficient of each photodiode for the reference 30 and active 34 detectors may result of gas concentration measurements errors that must be compensated. This invention comprises a method to measure the drift of the optoelectronic components and compensate for said measured drift. The compensation method is described in the preferred embodiment described and as shown at FIG. 1.

The method uses an emitter 24 that provides light emission outside of the absorption spectrum of the gas of interest, also referred as the optical compensation signal, an injection beam splitter 22 injecting the said optical compensation signal into the optical path of the absorption chamber 11. The method may include an antireflection cavity 26 adapted to absorb the useless signal passing through the beam splitter 22 that has not been injected in the optical path. As examples, the emitter 24 may be an heterostructure semiconductor laser, a vertical-cavity surface-emitting laser or a LED. As an example, and as a preferred embodiment, the emitter 24 is made with a 380 nm collimated LED. The injection beam splitter 22 is typically made of ultraviolet transmitting material, with or without coated surfaces, such as UV fused silica. It may be shaped as a right-angle prism, a pentaprism or other geometrical forms that split or divide the incident light in two beams. For example, and as a preferred embodiment, the injection beam splitter 22 may comprise a 1.5 mm thickness uncoated UV fused silica window having 4% of reflectivity on each surface.

The optical compensation signal is detected by both the reference 30 and the active 34 detectors. The microprocessor 62 calculates the ratio of the optical compensation signal falling on the active detector 34, also referred as C_{T}, to the optical compensation signal falling on the reference detector 30, also referred as C_{R}. The microprocessor 62 uses the compensation coefficient α to compensate for the short and long-term drift of optoelectronic components.

$$\text{α=} {\text{C}}_{\text{T}} / {\text{C}}_{\text{R}}$$

The compensation coefficient α₀ which is equivalent to α but referring to the ratio of the compensation signal C_{T} / C_{R} under 0 ppm gas concentration.

$${\text{α}}_{0} \text{=} {\text{C}}_{\text{T0}} / {\text{C}}_{\text{R0}}$$

The UV transmitted intensity I_{T} is now related to the UV incident intensity I_{R} by the following corrected Beer-Lambert law:

$${\text{I}}_{\text{T}} = \left(\text{α} / {\text{α}}_{0}\right) {\text{ρ}}_{0} {\text{I}}_{\text{R}} {\text{e-K}}_{\text{C}}$$

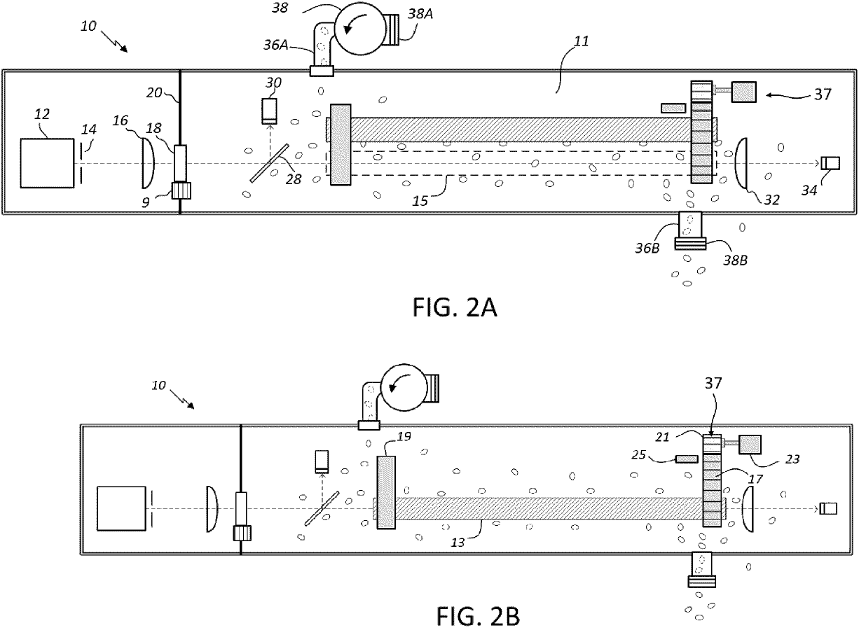

Referring now to FIGS. 2A and 2B, a second embodiment of a system and method to periodically change the absorption percentage in the absorption path by the mean of inserting a transparent material 13 in the optical path is illustrated. Broadly, the transparent material 13 must not interact with the gas of interest and may be for example a light transmitting pipe 13, typically a glass pipe. The measurement method uses the flashlamp 12 and the optical bandpass filter 18 to generate the optical compensation signal instead of the emitter 24 such as described in the first embodiment. The optical compensation coefficient α is then measured when the light pipe 13 is across the optical absorption path FIG. 2b.

The equations eq(4) and eq(5) are valid for both the optical compensation signal generated by the emitted source 24 in the first embodiment and by the flashlamp/bandpass filter source presented in the second embodiment when the light pipe 13 is across the optical absorption path. The incident and transmitted intensity, I_{R} and I_{T}, are measured when the light pipe 13 is not across the optical absorption path FIG. 2A.

The light pipe 13 may be made of material not sensitive to the gas of interest and may have a cross-section shaped as a circle, a square, a hexagonal or any geometrical shape surrounding the optical beam in the absorption path. For example, and as a preferred embodiment, the light pipe 13 may be made of an ultraviolet transmitting material such as UV fused silica or quartz. The input and/or output surfaces may be flat and/or have a small bevel angle. The light pipe 13 may also have an antireflection coating on both input and output surfaces. In one embodiment, the light pipe 13 may be fabricated with an uncoated UV-fused silica rod of 25 mm diameter cross section with an input and output bevel angle of 8 to 12 degrees.

The light pipe 13 is maintained parallel or slightly tilted compared to the optical axis by two cylinders 17 and 19 each having two holes, one for supporting the light pipe 13 and another hole 15 for allowing the UV light to pass through the absorption path when measuring the gas concentration. The light pipe 13 may be periodically inserted into the optical absorption path to measure the compensation coefficient α.

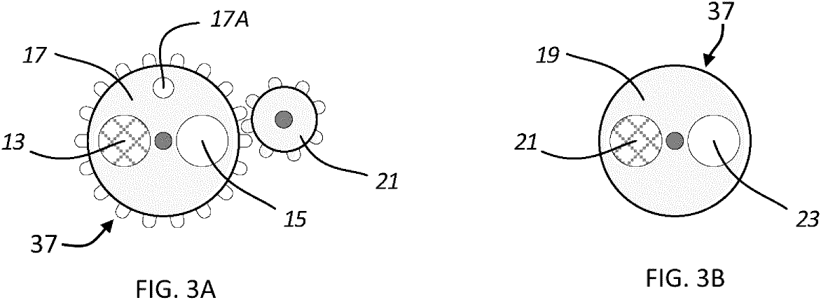

As shown on FIG. 3A, the cylinder 17 is shaped as a toothed wheel. In such embodiment, the cylinder 17 may be rotatably coupled to the gear 21. The cylinder 17 may further comprise a small hole or aperture 17A used as a mechanical reference for the position encoder 25. In the embodiment shown a FIG. 3B, the cylinder 19 comprises two holes, one being used as a rotation reference for the gear 21 and an the other to be used with an actuator 23. For example, and as a preferred embodiment, the light pipe 13 is inserted into the optical absorption path by means of the actuator 23, which may be for example a stepping motor or any other electromechanical component such as magnetic solenoid with or without compression spring. The system 10 may further comprise a subsystem 37 capable of periodically moving the light pipe 13 in and out of the optical absorption path. The subsystem 37 generally comprises the cylinders 17 and/or 19, the gear 21, the position encoder 25, the actuator 23 and any other mechanical subsystem.

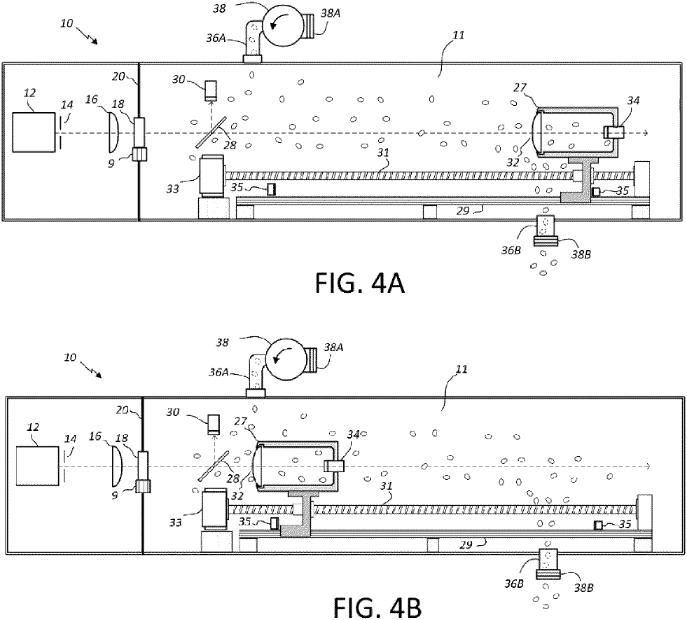

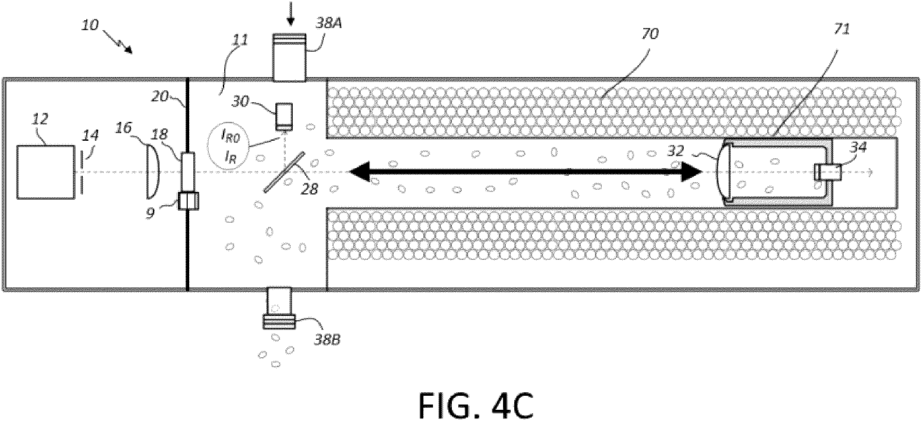

Referring now to FIG. 4A and FIG. 4B, a third embodiment of a method and system for measuring the gas concentration by periodically shortening the length of the optical absorption path is illustrated. In such embodiment, the length of the absorption path may be reduced or expanded by moving an optomechanical assembly 27 towards the beam splitter 28. The optomechanical assembly 27 generally comprises the active detector 34 and the focusing lens 32. The active detector 34 is mounted at the focal point of the lens 32 on the optomechanical assembly 27. The proposed measurement method uses the flashlamp 12 and the optical bandpass filter 18 to generate the optical compensation signal instead of the emitter 24 such as the one used in the first embodiment. Understandably, the optical compensation coefficient α is measured when the optomechanical assembly 27 is close to the beam splitter 28 in the collimated optical beam.

Referring now to FIG. 4C, in another embodiment, the optomechanical assembly 27 comprises two mating portions. The first portion is slidingly moveable within the second portion to change the length of the light path. In some embodiments, the first portion is a first cylinder slidingly moving within a second cylinder having a diameter slightly larger than the diameter of the outer surface of the first cylinder. The outer portion of the first cylinder may comprise threads mating with threads within the inner surface of the second cylinder. By rotating one cylinder in relation to the other, the length of the light path is increased or reduced. Typically, the active photodetector 34 and focusing lens 32 are mounted within the first cylinder, the first having an aperture at one end to allow the beam of light to pass through the assembly toward the active detector 34.

Still referring to FIG. 4C, the system 10 comprises a magnetic carriage or slider 71. The active photodetector 34 and focusing lens 32 are mounted to the magnetic carriage 71. The system 10 further comprises a magnetic winding, typically surrounding the inner cylinder. As shown, the carriage 71 may slide toward the light source 12 or away from the light source 12 when the magnetic winding 70 generates a magnetic field. Understandably, the direction of the movement of the carriage 71 may be adapted depending on the positioning of the magnetic winding and on the polarity of the magnetic carriage 71. The equations eq(4) and eq(5) are valid for both the optical compensation signal generated by the emitted source 24 in the first embodiment and by the flashlamp/bandpass filter source in the second and third embodiments when the light pipe 13 is in the optical absorption path or when the optomechanical assembly 27 is close to the beam splitter 28, respectively. When the optomechanical assembly 27 is away from the beam splitter 28, the absorption path is expanded or longer. The incident and transmitted intensities, I_{R} and I_{T}, are measured with the absorption path being expanded.

As an example, and as a preferred embodiment, the optomechanical assembly 27 may be attached to a linear rail 29 parallel to the optical axis. The optomechanical assembly 27 comprising the active detector 34 and the lens 32 is moved towards or away from the beam splitter 28. The assembly 27 is generally moved using a mechanical subsystem which may comprise a linear rail 29 positioned parallel to the optical axis, a captive worm screw 31 in a stepping motor 33 and two mechanical stops 35. In such embodiment, the rotation of the stepping motor moves the optomechanical assembly 27 on the rail 29 according to the direction of rotation of the stepping motor 33. Two physical positions programmed by two mechanical stops 35 define the position of the mechanical assembly 27 for both measuring the optical compensation coefficient α and the incident and transmitted intensity, I_{R} and I_{T} respectively. The compensation signal C_{T} is sampled on the active detector 34 when the active detector 34 is close to the beam splitter 28, thus when the absorption path is short. The transmitted intensity I_{T} is sampled on the active detector 34 when the active detector 34 is far from the beam splitter 28. Thus, when the absorption path is long. Other mechanical systems may be used to move the optomechanical assembly 27, such as belt driver linear actuator, rod-style actuator, or linear servo.

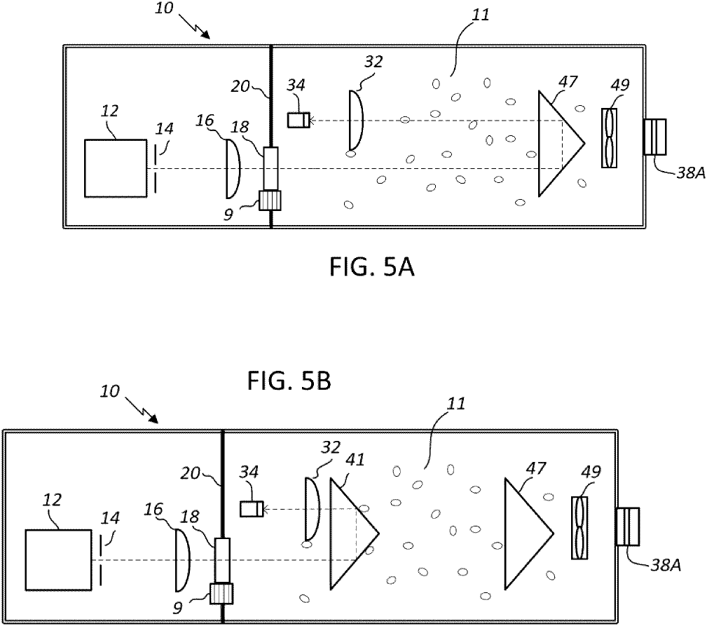

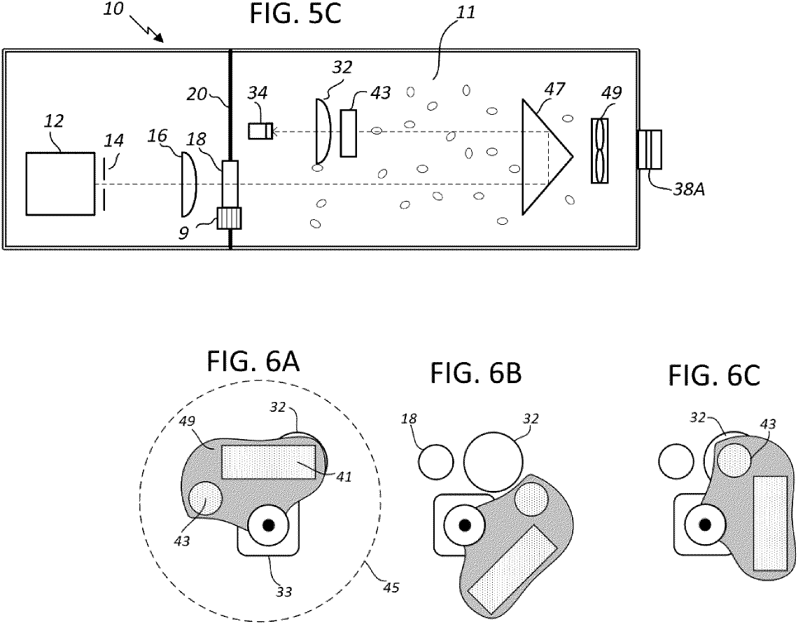

In a fourth embodiment and referring now to FIGS. 5A to 5C and FIGS. 6A to 6C, a system and method for measuring the gas concentration with a single detector by periodically reducing the length of the absorption path is provided. The long absorption path illustrated in FIG. 5A comprises and optical element 47 which deflect the optical beam towards the focusing lens 32. The active detector 34 is mounted at the focal point of the lens 32. The proposed measurement method uses the flashlamp 12, the lens 16 and the optical bandpass filter 18 to generate a collimated UV beam which will be absorbed by the gas of interest proportionally to its concentration level.

The length of the absorption path may be shortened by inserting an optical element 41 into the collimated optical beam which may then deflect the signal towards the focusing lens 32 so that the energy measured by the active detector 34 is stronger than the energy measured in the long absorption path. The optical element 41 may also be a pivoting element allowing a first position in which the signal is deflected and a second position in which the signal is not deflected.

The Beer-Lambert law eq(2) is applied to calculate the gas concentration based on the ratio of the measured transmitted intensity I_{T} to the measured incident intensity I_{R}. The incident intensity, I_{R} is measured when the optical element 41 is inserted into the optical beam. The transmitted intensity I_{T} is measured when the optical element 41 is not present into the optical beam or pivoted not to deflect the signal. The correction coefficient ρ₀ calculated in eq(3) compensates for the inherent differential optical intensity of UV light falling on the active 34 detectors at 0 ppm gas concentration for incident I_{R0} and transmitted I_{T0} intensities.

As an example, and as a preferred embodiment, the optical elements 41 and 47 may be prisms made of transparent material such a synthetic fused silica, but any other ultraviolet-transparent material such as quartz, CaF₂ or MgF₂ may be used. The incident angle on the prism input surface is preferably less than the Brewster angle for complete internal reflection on the opposite surfaces in the UV band. Other optical element 41 and 47 may be used to deflect the optical beam, such as a corner cube or aligned mirrors.

Now referring to FIGS. 6A to 6C, different positions of the optical element 41 are illustrated. The optical element 41 may be inserted in the optical beam. The system 45 comprises a blade 49 which supports the optical element 41 and an actuator 33 to rotate the blade 49, so that the optical element 41 can be placed into or removed from the optical beam. FIG. 6A shows the position of the blade 49 when measuring the incident signal I_{R} or I_{R0}, wherein the optical beam may be deflected by an angle of 180 degrees towards the focusing lens 32 to create a short absorption path.

FIG. 6B shows the case where the blade 49 is rotated clockwise to remove the optical element 41 from the optical beam. In this case, the optical beam is free to be reflected by the optical element 47 to create a long absorption path for the transmitted signal I_{T} or I_{T0}.

In another embodiment of the present invention, a method to periodically recalibrate the system 10 without the need of a known gas concentration in the absorption chamber is provided. The method comprises inserting a semi-transparent material 43 in the optical path such that the transmitted intensity is attenuated by a known value corresponding to a precise gas concentration. This method implies that the system has been previously characterized with a precise concentration gas sample compared to the attenuation element 43. For example, and as a preferred embodiment, the attenuation element 43 may be a simple window having 4% reflection on each surface but could be any other semi-transparent optical element in the UV band such as neutral density filter, band pass filter or broadband filter with or without coating. This embodiment is illustrated in FIG. 6C wherein the attenuation element 43 is inserted into the optical beam in front of the focusing lens 32. The attenuation element 43 acts like a gas whose concentration value is known and is used for the system 10 to do a recalibration without the need of any gas in the absorption chamber 11.

The long-term stability of the system 10 depends on the stability of certain element such as the bandpass filter 18. It is well known that bandpass filters change in centered wavelength and are influenced by the ambient relative humidity. Such elements will influence the long-term stability of the gas analyser 10. To avoid this drift, a system and method to maintain the bandpass filter 18 at a temperature higher than the internal temperature of the system 10 is provided. The method may allow for the temperature of the filter 18 to be higher than the dew point temperature of gases present in the system 10. A system may accordingly comprise a heating element 9 and a temperature sensor, not shown, in physical contact with the filter 18 to raise its temperature above the internal ambient temperature of the system 10.

. Now referring to FIG. 11, a bloc diagram illustrates both the reference 30 and active detectors 34. In a preferred embodiment, both circuits 30 and 34 are similar and differ only by their specific analog gain. The first stage of the detector circuits comprises a photodetector 40 responding to ultraviolet light down to 190 nm and a transimpedance amplifier 42 that converts the photocurrent into a voltage. The photodetector 40 may be any suitable light sensitive device but, in the illustrated embodiment, is preferably an ultraviolet enhanced silicon photodiode having a response of 0.05 A/W in the band of interest. The intensity measured by the photodiode 40 is substantially proportional to the incoming light. The transimpedance gain is set by the resistor R_{G} selected in accordance with the light intensity falling on the photodiode 40. The second stage of the detector circuits comprises an integrator 46 using the capacitor C_{I} to integrate the light pulse emitted by the flashlamp or by the 380 nm LED. The output of the integrator may thus provide a signal to the analog-to-digital converter 50. The integrator may be reset using the switch SW4. After the pulse is integrated, the capacitor holds the integrated signal while the said signal is measured by the analog-to-digital converter 50. The switch SW1 isolates the integrator circuit from the transimpedance amplifier to avoid any leakage from the integration capacitor C_{I} after light pulse reach zero and during the conversion process.

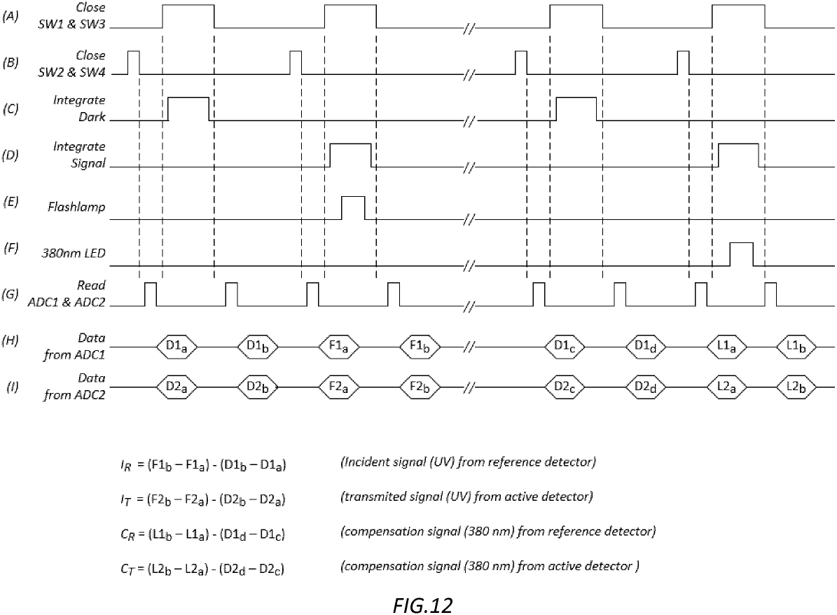

Referring the now to FIGS. 12 to 15, measurements of intensities and optical compensation signals are presented, such as incident and transmitted intensities and the optical compensation signals measured by both the active 34 and the reference 30 detectors. Referring to FIG. 12, a timing diagram of measuring process of an embodiment of this invention is shown. Before every integration period, each integration capacitor C_{I} is discharged by the electronic switch SW2/SW4 (B) to remove unwanted remaining charges. The acquisition process follows a double-correlated-sampling and hold technic to remove unwanted switching noise injected by the resetting switch SW2/SW4. These levels correspond to D2a/F2a and D1a/F1a for the reference and active detector, respectively. With the flashlamp 56 off, each integration capacitor accumulates the background and dark noise (C) by closing the switch SW1/SW3 (A). The real contribution of background and dark noise is the subtraction of (D1b-D1a) and (D2b-D2a) for the reference and active detector, respectively. In a second sampling sequence, with the flashlamp 56 pulsed (E), the contribution of the flashlamp (D) is measured by (F1b-F1a) for the reference detector and (F2b-F2a) for the active detector. The respective background and dark noise are finally subtracted to the contribution signal of flashlamp to get the real value of the incident I_{R} and transmitted I_{T} intensity. A similar sequence follows to measure the optical compensation signal C_{R} and C_{T} emitted by the emitter 24 and integrated by the reference detector and active detector, respectively. The last sampling integration sequence is almost identical to the first one except that the light source is replaced by the emitter 24 instead of the flashlamp 56.

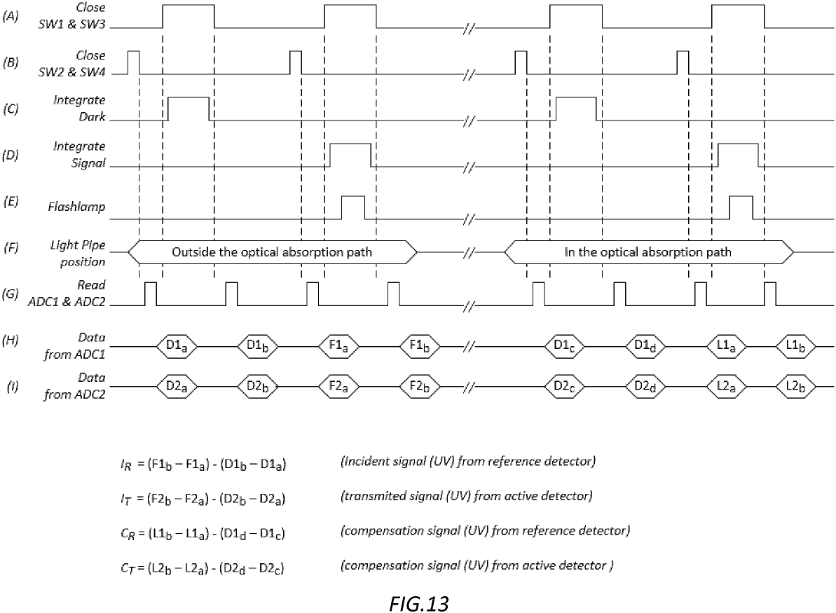

FIG. 13 shows a timing diagram of a method according to the second embodiment of the present invention when the compensation signal is generated by the flashlamp/bandpass filter when the light pipe 13 is in the absorption path. The timing diagram is almost identical to that of FIG. 12, except that the 380 nm LED source 24 in (F) is substituted by the insertion of the transparent optical rod 13 in the absorption path. All equations are valid for both the first and second methods of the first and second embodiments, respectively.

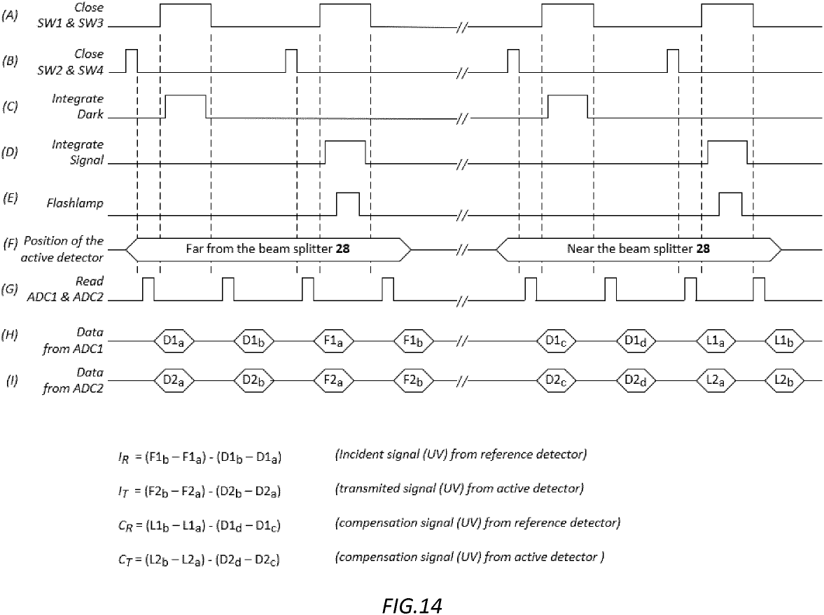

FIG. 14 shows the timing diagram of the third embodiment of the present invention when the compensation signal is generated by the flashlamp/bandpass filter when the optomechanical assembly 27 is close to the beam splitter 28. The timing diagram is almost identical to that of FIG. 12, except that the 380 nm LED source 24 in (F) is substituted by moving the optomechanical assembly 27 near or adjacent to the beam splitter 28. All equations are valid for both the first, the second and the third methods of the first, the second and the third embodiments, respectively.

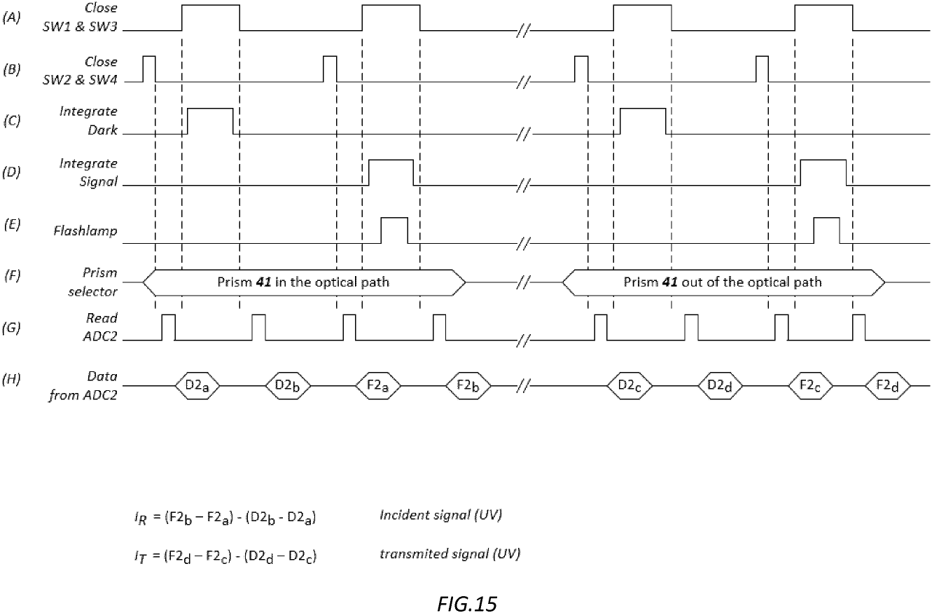

FIG. 15 shows the timing diagram of the fourth embodiment of the present invention when the incident signal I_{R} is measured when the optical element 41 is in the optical path to create a short absorption path. The transmitted signal I_{T} is then measured for a long absorption path when the prism 41 is out of the optical path. The timing diagram is almost identical to that of FIG. 12, except that only one detector is required to measure the incident and the transmitted signal. The equations described before are valid, but the compensation coefficients α and α₀ equal 1 because of the single detector architecture.

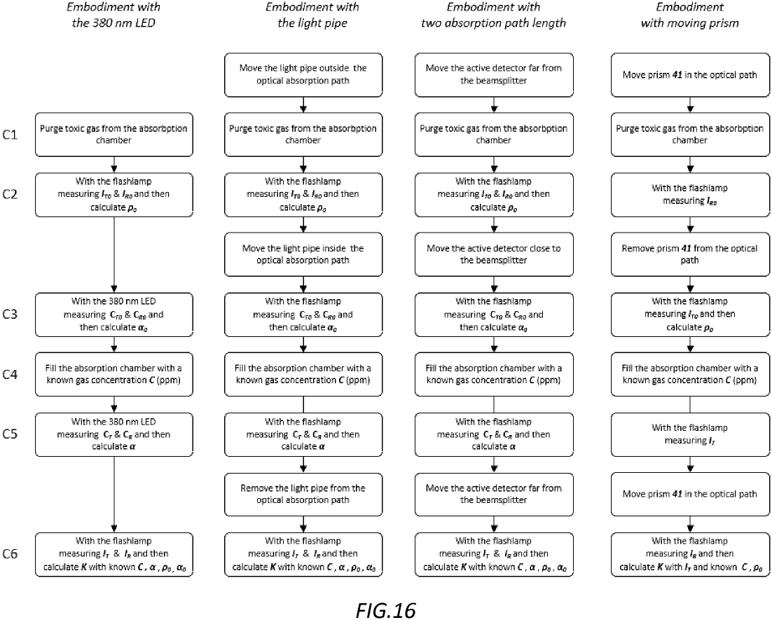

Now referring to FIG. 16, a flow diagram illustrates the calibration methods for each of the selected embodiment. Each of the calibration sequence performs the step under program control for sequentially calculating the coefficient ρ₀, α₀ and K. The step C_{I} generally ensures that the absorption chamber is free of toxic gas or gas of interest that may interfere with the UV light in the band of interest. The incident I_{R0} and transmitted I_{T0} intensity in 0 ppm gas concentration condition are measured C2 by the reference 30 and active 34 detector respectively and coefficient ρ₀ is then calculated. In C3, the optical compensation signal is emitted by the emitter 24 or by combination of the flashlamp & bandpass filter. The compensation signal C_{R0} is measured on the reference detector 30 and the compensation signal C_{T0} is measured on the active detector 34 to provide the compensation coefficient ao. At C4, the absorption chamber is filled with a known ppm concentration of the gas to be analyzed. At C5, the optical compensation signal is emitted by the emitter 24 or by the combination of the flashlamp & bandpass filter.

The compensation signal C_{R} is measured on the reference detector 30 and the compensation signal C_{T} on the active detector 34 to provide the compensation coefficient α of the known ppm concentration. The coefficient K is finally calculated in C6 with eq(7) by measuring the incident intensity I_{R}, and the transmitted intensity I_{T} with the flashlamp. The embodiment with the moving prism architecture is a simplified version of the ones having two detectors. There is no compensation signal and the compensation coefficients α and α₀ in all equation must be replace by 1 for both.

$$K = \text{-} \left(1 / C\right) l n \left({I}_{T} {α}_{0} / \left({I}_{R} α {ρ}_{0}\right)\right)$$

The gas concentration is simply measured by rewriting eq(7) as follow.

$$C = \text{-} \left(1 / K\right) l n \left(I T {α}_{0} / \left({I}_{R} α {ρ}_{0}\right)\right)$$

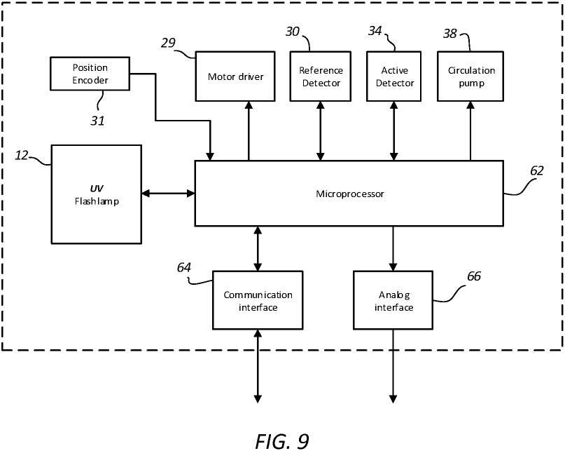

Referring now to FIGS. 8 and 9, a diagram illustrates the system components described above in relation to the microprocessor 62. A communication interface 64 provides a path to receive control commands from an external master controller, to read the gas concentration or to execute the calibration process. For example, and as a preferred embodiment, the communication interface 64 is an EIA-485 interface but it is well understood that it may be any type of digital physical interface. An analog interface 66 provides a way to read the gas concentration by common analog signals such as 0-10 V or 4-20 mA but not restricted to those, it may be for example a pulse-width modulation (PWM) signal, an optical link, or an RF interface.

Certain aspects of the present invention include process steps and sequences described herein in the form of a sequence. It should be noted that the sequence necessary to measure the gas concentration could be embodied in software, firmware, or hardware, and when embodied in firmware, could reside and be operated from different platforms used by a variety of operating systems.

An advantage of the present invention is to provide an UV non-dispersive gas analyser that is insensitive for the presence of interfering gas, such as CO₂, O₂, CO, CH₄ and /or water vapor. According to another embodiment, the present invention provides the advantage to be insensitive to the light emission spectrum degradation due to lamp aging effects provided. Finally, this invention further comprises methods to compensate for short and long terms drift of the components. This method may further comprise a gas analyser which does not require further calibration and where the measurement of the gas concentration is very stable over temperature and lifetime differences.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1) A system for measuring concentration of a gas, the system comprising:
an absorption chamber comprising an air inlet and an air outlet;
an active photodetector within the absorption chamber to measure a light radiation;
a controllable active light source emitting an ultraviolet light beam within an absorption spectrum of the gas along an absorption path toward the active photodetector;
an optical bandpass filter between the active light source and the active detector; and
a reference photodetector positioned to measure the light beam of the active light source entering in the absorption chamber.

2) The system according to claim 1, the active light source being a controllable ultraviolet “arc type”.

3) The system according to claim 1, the system further comprising:
a first interface for sampling a signal outputted by the active photodetector;
a second interface for sampling the signal outputted by the reference photodetector;
a computerized device connected to the first and second sampling interfaces, the computerized device being configured to calculate the concentration of the gas as a function of light absorption based on a ratio of the output signals of the reference photodetector and of the active photodetector.

4) The system according to claim 1, the system further comprising an optical element between the active light source and the absorption chamber used to generate a collimated light beam within the absorption chamber.

5) The system according to claim 4, the system further comprising a lens adjacent to the active detector for collimating the light emitted in the absorption chamber to the active photodetector.

6) The system according to claim 1, the system further comprising a heating element adapted to heat the optical bandpass filter at a temperature higher than the dew point temperature of the gas.

7) The system according to claim 1, the system further comprising a beam splitter to direct a portion of the light beam emitted in the absorption chamber towards the reference detector.

8) The system according to claim 7, the system further comprising a controllable reference light source emitting a light beam outside of the absorption spectrum of the gas, the light beam being measurable by the active and the reference photodetector.

9) The system according to claim 8, the system further comprising a drift compensation mechanism.

10) The system according to claim 9, the drift compensation mechanism being configured to adjust the measurement of the gas concentration using a proportional ratio of drift values measured by the active photodetector and the reference photodetector since the last calibration.

11) A system for measuring concentration of a gas, the system comprising:
an absorption chamber comprising an air inlet and an air outlet;
an active photodetector within the absorption chamber to measure light radiation;
a controllable light source emitting an ultraviolet light beam within an absorption spectrum of the gas along an absorption path toward the active photodetector;
an optical bandpass filter between the light source and the active detector; and
a device to change the length of the light path in the absorption chamber between the light source to the active photodetector.

12) The system according to claim 11, the system further comprising:
a first interface for sampling the ultraviolet light beam detected by the active photodetector;
a second interface for controlling position of the device to change the length of the light path within the absorption chamber;
a computerized device connected to the first and the second interfaces, the computerized device being configured to calculate the concentration of the gas as a function of light absorption measured by the signal ratio before and after changing the length of the light path through the absorption chamber.

13) The system according to claim 11, the device to change the length of the light path being a light pipe insertable in the light path yet removable from the light path between the light source and the active photodetector.

14) The system according to claim 11, the device to change the length of the light path between the light source and the active photodetector being a support movable toward and away from the light source, the active photodetector being mounted to the movable support.

15) The system according to claim 14, the support being moved using an electromotive force.

16) The system according to claim 14, the support being a carriage.

17) The system according to claim 14, the support comprising two mating portions, the first portion slidingly moving within the second portion to change the length of the light path.

18) The system according to claim 11 wherein the light source and the active photodetector are oriented in the same direction toward the absorption chamber, the device to change the length of the light path comprising:
a first reflecting member in the absorption chamber returning the ultraviolet light beam to the active photodetector setting a long light path; and
a second reflecting member insertable between the first reflecting member and the light source to set a short light path.

19) The system according to claim 11, the system further comprising an optical element between the light source and the absorption chamber used to generate a collimated light beam within the absorption chamber.

20) The system according to claim 19, the system further comprising a lens adjacent to the active detector for collimating the light emitted in the absorption chamber to the active photodetector.

21) The system according to claim 11, the system further comprising a heating element adapted to heat the optical bandpass filter at a temperature higher than the dew point temperature of the gas.

22) A method for measuring a concentration of a gas present in an absorption chamber, the method comprising:
emitting a light beam through the absorption chamber at a wavelength absorbed by the gas.
measuring a reference intensity of the emitted light entering in the absorption chamber;
measuring an active intensity of the emitted light after passing through the gas in the absorption chamber at a predetermined distance of the emission of the light; and
calculating the gas concentration based on the ratio of the of measured active intensity and of the measured reference intensity.

23) The method of claim 22, the method further comprising filtering the emitted light entering the absorption chamber at a wavelength absorbed by the gas.

24) The method of claim 22, the measuring of the reference intensity being performed by a first photodetector and the measuring of the active intensity being performed by a second photodetector.

25) The method of claim 22, the method further comprising deflecting a portion of the emitted light to measure the reference intensity.

26) A method for measuring a concentration of a gas present in an absorption chamber comprising a light path having a variable length between a light source and a photodetector, the method comprising:
reducing the length of the light path in the absorption chamber;
emitting a light beam through the absorption chamber at a wavelength absorbed by the gas in the reduced light path;
measuring a reference intensity of the emitted light in the reduced light path;
increasing the length of the light path in the absorption chamber;
emitting the light beam through the absorption chamber at a wavelength absorbed by the gas in the increased light path;
measuring an active intensity of the emitted active light beam in the increased light path; and
calculating the gas concentration based on the ratio of the measured intensities from the reduced light path and the increased light path.

27) The method of claim 26, the reducing of the length of the light path in the absorption chamber further comprising inserting into the emitted light beam a light pipe inert to the gas.

28) The method of claim 26, the reducing of the length of the light path in the absorption chamber further comprising moving the light source and the photodetector toward one another.

29) The method of claim 26, the photodetector and the light source being oriented in the same direction, the photodetector receiving the light beam through a first reflecting member, the reducing of the length of the light path in the absorption chamber further comprising placing a second reflecting member between the light source and the first reflecting member.

30) A method to correct for short and long terms drifts of the system of claim 8, the method further comprising:
turning off the active light source and the reference light source;
measuring a reference intensity when the active light source and the reference light source are turned off;
measuring an active intensity when the active light source and the reference light source are turned off;
turning on the reference light source through the absorption chamber at a wavelength outside of the absorption spectrum of the gas;
measuring a reference intensity when the reference light source is turned on;
measuring an active intensity when the active light source is turned on;
calculating a reference signal drift based on the difference between the measured reference intensities;
calculating an active signal drift based on the difference between the measured active intensities;
calculating a drift ratio of the reference signal drift and the active signal drift; and
correcting calculation of the gas concentration using the calculated drift ratio.
